(19) United States
(12) Patent Application Publication
(10) Pub. No.: US 2021/0069209 A1
(43) Pub. Date: Mar. 11, 2021

(54) NOVEL PHARMACEUTICAL COMPOSITIONS AND METHODS FOR MENOPAUSE RELATED ANXIETY AND DEPRESSION

(71) Applicant: LA PharmaTech Inc., Blacksburg, VA (US)

(72) Inventors: Jianmin Wang, Blacksburg, VA (US); Geping Cui, Beijing (CN)

(21) Appl. No.: 17/094,405

(22) Filed: Nov. 10, 2020

Related U.S. Application Data
(63) Continuation-in-part of application No. 16/884,459, filed on May 27, 2020.
Application No. 16/884,459 is a continuation-in-part of application No. 16/382,885, filed on Apr. 12, 2019.
Application No. 16/382,885 is a continuation-in-part of application No. 16/884,553, filed on May 27, 2020, now Pat. No. 10,898,493.
Application No. 16/884,553 is a continuation-in-part of application No. 16/382,885, filed on Apr. 12, 2019.
Application No. 16/382,885 is a continuation-in-part of application No. PCT/US20/34735, filed on May 27, 2020.
Application No. PCT/US20/34735 is a continuation-in-part of application No. 16/382,885, filed on Apr. 12, 2019.

(51) Int. Cl. A61K 31/5517; A61K 31/55; A61K 9/00; A61P 25/22; A61P 5/24

(52) U.S. Cl. CPC A61K 31/5517; A61K 31/55; A61P 5/24; A61P 25/22; A61K 9/0053

(57) ABSTRACT

Pharmaceutical compositions comprising azelastine or a pharmaceutically acceptable salt of azelastine and alprazolam are disclosed. Methods of using the pharmaceutical compositions for treating perimenopausal or menopausal patients, such as patients suffering from, experiencing, exhibiting and/or having one or more symptoms of anxiety or depression, are also disclosed.

### CROSS-REFERENCE TO RELATED APPLICATIONS

The present application is a Continuation-in-Part application of U.S. patent application Ser. No. 16/884,459, which was filed on May 27, 2020 and which published as U.S. Application Publication No. 2020/0323876 on Oct. 15, 2020. The present application is a Continuation-in-Part application of U.S. patent application Ser. No. 16/884,553, which was filed on May 27, 2020 and which published as U.S. Application Publication No. 2020/0323877 on Oct. 15, 2020. The present application is a Continuation-in-Part application of International Application No. PCT/US20/34735, which was filed on May 27, 2020. The '459 Application, the '553 Application, and the '735 Application are each a Continuation-in-Part application of U.S. patent application Ser. No. 16/382,885, which was filed on Apr. 12, 2019 and which published as U.S. Application Publication No. 2020/0323867 on Oct. 15, 2020. The disclosures of these applications are hereby incorporated by reference herein in their entireties.

### FIELD OF THE INVENTION

The invention relates to the field of practical medicine, namely, the use of pharmaceutical compositions for alleviating manifestations of anxiety and depression from patients during perimenopause and menopause.

### BACKGROUND OF THE INVENTION

Psychiatric disorders, including anxiety and depression, affect more than one in ten people globally (10.7%) as reported by the Institute for Health Metrics and Evaluation in their flagship Global Burden of Disease study in 2017. During perimenopausal and menopausal periods, the decrease in estrogen may lead to anxiety and depression. Hot flashes and insomnia during those transitions may also cause emotional distress. Studies have shown that women in the perimenopausal and menopausal periods are more likely to experience panic attacks and other anxiety and depression symptoms than other women of the same age who are either pre- or postmenopausal. Studies also report that 23 percent of women experience symptoms of anxiety alone during perimenopause and an even higher percent of women experience depression, including major depression.

Treatments for anxiety and depression for patients experiencing perimenopause or menopause may include selective serotonin reuptake inhibitors (SSRIs), such as citalopram (Celexa); selective serotonin & norepinephrine inhibitors (SNRIs or SSNRIs), such as desvenlafaxine (Khedezla); tetracyclic antidepressants of noradrenergic and specific serotonergic antidepressants (NaSSAs), such as Remeron; tricyclic antidepressants, such as Elavil; monoamine oxidase inhibitors (MAOIs), such as isocarboxazid (Marplan); and benzodiazepines, such as alprazolam (Xanax). These drugs do carry a range of side effects, such as addiction, intolerance, loss of effectiveness, inciting violent or self-destructive actions, fatigue, and drowsiness along with nausea, increased appetite and weight gain, loss of sexual desire and other sexual problems, insomnia, dry mouth, blurred vision, and so on. Studies do find that hormone therapies are effective for perimenopausal and early postmenopausal women who experience symptoms of depression. However, hormone therapy brings its own set of risks, such as a greater chance of blood clots, stroke, and breast cancer.

Clinically, new treatments that provide tremendous improvement and significant impact to the management of anxiety and depression for patients experiencing perimenopause or menopause, such as treatments highly effective for long term use and with fewer adverse effects, are highly and urgently needed.

Estrogen, as one of the primary hormones, has an important role in the health of women. The ovaries make most of the estrogen. In women, estrogen circulates in the bloodstream and binds to estrogen receptors on cells in targeted tissues, affecting not only the breasts and uterus, but also the brain. Women's estrogen levels begin to fluctuate and fall during perimenopause and menopause. The changing levels of estrogen create many health problems, including anxiety and depression.

The onsets of perimenopause and menopause in women cause changes in the composition and function of the immune system. Estrogen has been suggested to function as a potent anti-inflammatory factor, and the reduction of estrogen as a result of perimenopause and menopause activates systemic adaptive and innate immune responses. Perimenopause and menopause coincide with higher levels of circulating interleukins (IL-6, IL-4, IL-2) and tumor necrosis factor (TNF), and the increases in these factors have been shown to be reversed by hormone therapy. Increasing evidence indicates that inflammatory processes can cause and contribute to the development of anxiety and depression.

Inflammation can be defined as one of the immune responses for protecting living organisms from damage. The latest advancements in neurobiological research provide increasing evidence that inflammatory and neurodegenerative pathways play a relevant role in depression and anxiety. Preclinical and clinical studies on depression and anxiety highlighted an increased production of inflammatory markers, such as interleukin (IL)-1, IL-6, tumor necrosis factor (TNF)-α and interferon (INF)-α and γ, and overactivated inflammatory signaling pathways including nuclear factor kappa B (NF-κB). Other studies show that acute and chronic administration of cytokines or cytokine inducers were found to trigger depressive and/or anxiety symptoms. According to the cytokine hypothesis, depression and anxiety would be due to a stress-related increased production of pro-inflammatory cytokines that, in turn, would lead to increased oxidative and nitrosative brain damage and consequent reduced availability of tryptophan and serotonin (5-HT). Cytokines would also play a role in the onset of the glucocorticoid resistance, underlying the overdrive of the hypothalamic-pituitary-adrenal axis. Therefore, activation of the inflammatory and neurodegenerative pathways would lead to the brain damage observed in depression and/or anxiety through both reduced neurogenesis and increased neurodegeneration.

Azelastine is classified pharmacologically as a second-generation antihistamine and is a relatively selective, non-sedative, competitive antagonist at H₁ receptors for treatment of allergic rhinitis and asthma. But, more uniquely, its inhibition of inflammatory mediators and its mast cell stabilizing effects, in addition to its antihistaminic activity, place it among the new generation of dual-acting anti-inflammatory drugs. Its ability to modify several other mediators of inflammation, such as IL-1, IL-6, TNF-α and INF-α, and to reduce overactivation of NF-κB inflammatory signaling pathway might contribute to its mechanism of action of potential treatment of anxiety and depression associated with perimenopause and menopause. In vitro and in vivo studies, as well as clinical trials, support the dual effects of direct inhibition and stabilization of inflammatory cells. In vitro data indicate that azelastine's affinity for inhibition of mast cell degranulation may also decrease the release of other inflammatory mediators, including leukotrienes and interleukin-1β, among others. Preclinical studies show that azelastine also directly antagonizes other mediators of inflammation, such as tumor necrosis factor-α, leukotrienes, endothelin-1, and platelet-activating factor.

Alprazolam, the 19th most prescribed drug with 27 million prescriptions in the U.S. in 2019, is a benzodiazepine derivative that is currently used in the treatment of generalized anxiety, panic attacks, and depression. Alprazolam binds non-selectively to the gamma-amino butyric acid A (GABAA)-benzodiazepine receptor complex. At the receptor complex, alprazolam facilitates the binding of GABA and increases the influx of chloride ions. The presence of GABA, in turn, inhibits the action of several connected brain structures, resulting in a general slowing of brain activity. Further, the activated GABA system interacts with other neurotransmitter systems, including noradrenergic, serotonergic, cholinergic, and opioidergic systems. Alprazolam's interactions with the serotonergic and noradrenergic pathways to the limbic system and brain stem structures (e.g., locus coeruleus) contribute to its clinical effectiveness in the treatment of anxiety and depression. Alprazolam has a fast onset of symptom relief and is significantly superior to placebo. However, its adverse effects, such as cognitive and psychomotor effects, drowsiness, sedation, withdrawal and rebound effects, limit its long-term therapeutic applications. One of the most critical adverse effects of alprazolam is impairment in performance of a variety of patient skills due to its effect on psychomotor and memory functioning.

Therefore, a unique combination of azelastine (antihistamine agent with anti-inflammatory activities) with alprazolam (GABA-benzodiazepine receptor complex agonist) would potentially be, in terms of working through multi-mechanisms of actions, effective treatments for anxiety and depression associated with perimenopause and menopause.

### SUMMARY OF THE INVENTION

The present invention includes a pharmaceutical composition that comprises two active pharmaceutical ingredients. This pharmaceutical composition comprises the first active ingredient that is azelastine or a pharmaceutically acceptable salt of azelastine and the second active ingredient that is alprazolam.

In some embodiments of this invention, the pharmaceutically acceptable salt of azelastine in the pharmaceutical composition is azelastine hydrochloride.

In some embodiments of this invention, azelastine hydrochloride (and/or other salt thereof) in the pharmaceutical composition is provided in an amount of about 1 mg to about 8 mg and alprazolam in an amount of about 0.2 mg to about 4 mg.

The present invention also includes an oral pharmaceutical dosage form of the pharmaceutical composition that is a solid, liquid, gel, or solution form.

The present invention further includes use of the composition, such as by oral dosage, through administration to perimenopausal or menopausal patients with anxiety or depression.

In some embodiments of this invention, an oral pharmaceutical dosage form of the pharmaceutical composition containing azelastine hydrochloride (and/or other salt thereof) in an amount of about 1 mg to about 8 mg and alprazolam in an amount of about 0.2 mg to about 4 mg is administered to perimenopausal or menopausal patients with anxiety or depression.

Embodiments include Aspect 1, which are pharmaceutical compositions comprising: azelastine or a pharmaceutically acceptable salt of azelastine; alprazolam; and one or more pharmaceutically acceptable excipients.

Aspect 2 is the pharmaceutical composition of Aspect 1, wherein the azelastine or the pharmaceutically acceptable salt of azelastine is present in the pharmaceutical composition in an amount in the range of about 1 mg to about 8 mg.

Aspect 3 is the pharmaceutical composition of Aspect 1 or 2, wherein the alprazolam is present in the pharmaceutical composition in an amount in the range of about 0.2 mg to about 4 mg.

Aspect 4 is the pharmaceutical composition of any of Aspects 1-3, wherein the alprazolam is present in the pharmaceutical composition in an amount in the range of up to about 4 mg.

Aspect 5 is the pharmaceutical composition of any of Aspects 1-4, wherein: the azelastine or the pharmaceutically acceptable salt of azelastine is present in the pharmaceutical composition in an amount in the range of about 1 mg to about 6 mg; and the alprazolam is present in the pharmaceutical composition in an amount in the range of about 0.2 mg to about 1 mg.

Aspect 6 is the pharmaceutical composition of any of Aspects 1-5, wherein the pharmaceutically acceptable salt of azelastine is azelastine hydrochloride.

Aspect 7 is the pharmaceutical composition of any of Aspects 1-6, wherein the pharmaceutically acceptable salt of azelastine is azelastine hydrochloride and is present in an amount in the range of up to about 8 mg.

Aspect 8 is the pharmaceutical composition of any of Aspects 1-7, wherein the azelastine hydrochloride is present in an amount in the range of about 1 mg to about 6 mg.

Aspect 9 is the pharmaceutical composition of any of Aspects 1-8, wherein the pharmaceutical composition is formulated as an oral pharmaceutical dosage form.

Aspect 10 is the pharmaceutical composition of any of Aspects 1-9, wherein the oral pharmaceutical dosage form is a solid form or a liquid form.

Aspect 11 is a method comprising: administering a pharmaceutical composition to a patient; wherein the pharmaceutical composition comprises azelastine or a pharmaceutically acceptable salt of azelastine and alprazolam, such as in effective amounts, wherein the effective amounts together are sufficient to treat one or more symptoms of anxiety or depression associated with perimenopause or menopause.

Aspect 12 is the method of Aspect 11, wherein the pharmaceutical composition is administered once or twice a day, or once every 2 or 3 or 4 days, to the patient, such as in an oral solid or liquid form.

Aspect 13 is the method of any of Aspects 11-12, wherein the pharmaceutical composition is administered for a period of at least 2 weeks.

Aspect 14 is the method of any of Aspects 11-13, wherein the azelastine or the pharmaceutically acceptable salt of azelastine is present in the pharmaceutical composition in an amount in the range of about 1 mg to about 8 mg.

Aspect 15 is the method of any of Aspects 11-14, wherein the alprazolam is present in the pharmaceutical composition in an amount in the range of up to about 4 mg.

Aspect 16 is the method of any of Aspects 11-15, wherein the alprazolam is present in the pharmaceutical composition in an amount in the range of about 0.2 mg to about 4 mg.

Aspect 17 is the method of any of Aspects 11-16, wherein the pharmaceutically acceptable salt of azelastine is azelastine hydrochloride and is present in the pharmaceutical composition in an amount in the range of about 1 mg to about 8 mg.

Aspect 18 is the method of any of Aspects 11-17, wherein: the azelastine or the pharmaceutically acceptable salt of azelastine is present in the pharmaceutical composition in an amount in the range of about 1 mg to about 8 mg; and the alprazolam is present in the pharmaceutical composition in an amount in the range of about 1 mg to about 4 mg.

Aspect 19 is a pharmaceutical composition comprising up to about 8 mg azelastine, or a pharmaceutically acceptable salt of azelastine, alprazolam, and one or more pharmaceutically acceptable excipients.

Aspect 20 is the pharmaceutical composition of Aspect 19, wherein the alprazolam is present in the pharmaceutical composition in an amount in the range of up to about 4 mg.

Aspect 21 is the pharmaceutical composition of Aspect 19 or 20, wherein the azelastine or the pharmaceutically acceptable salt of azelastine is present in the pharmaceutical composition in an amount 5 times that of the alprazolam present in the pharmaceutical composition.

Aspect 22 is the pharmaceutical composition of any of Aspects 19-21, wherein the azelastine or the pharmaceutically acceptable salt of azelastine is present in the pharmaceutical composition in an amount in the range of about 2 mg, and the alprazolam is present in the pharmaceutical composition in an amount in the range of about 0.4 mg.

Aspect 23 is the pharmaceutical composition of any of Aspects 19-22, wherein the pharmaceutically acceptable salt of azelastine is azelastine hydrochloride.

Aspect 24 is the pharmaceutical composition of any of Aspects 19-23, wherein the pharmaceutical composition is formulated as an oral pharmaceutical dosage form.

Aspect 25 is a method comprising administering a pharmaceutical composition to a perimenopausal or menopausal patient, wherein the pharmaceutical composition comprises azelastine, or a pharmaceutically acceptable salt of azelastine, and alprazolam.

Aspect 26 is the method of Aspect 25, wherein the pharmaceutical composition is administered once or twice a day, or once every 2 or 3 or 4 days to the patient in an oral solid or liquid form.

Aspect 27 is the method of Aspect 25 or 26, wherein the pharmaceutical composition is administered for a period of at least 2 weeks.

Aspect 28 is the method of any of Aspects 25-27, wherein the azelastine or the pharmaceutically acceptable salt of azelastine is present in the pharmaceutical composition in an amount in the range of about 1 mg to about 8 mg.

Aspect 29 is the method of any of Aspects 25-28, wherein the azelastine or the pharmaceutically acceptable salt of azelastine is present in the pharmaceutical composition in an amount that is 5 times the amount of alprazolam present in the pharmaceutical composition.

Aspect 30 is the method of any of Aspects 25-29, wherein the alprazolam is present in the pharmaceutical composition in an amount in the range of about 0.2 mg to about 4 mg.

Aspect 31 is the method of any of Aspects 25-30, wherein the azelastine or the pharmaceutically acceptable salt of azelastine is present in the pharmaceutical composition in an amount in the range of about 1 mg to about 6 mg.

Aspect 32 is the method of any of Aspects 25-31, wherein the azelastine or the pharmaceutically acceptable salt of azelastine is present in the pharmaceutical composition in an amount in the range of up to about 8 mg, and the alprazolam is present in the pharmaceutical composition in an amount in the range of up to about 4 mg.

Aspect 33 is a method comprising administering to a patient, who has exhibited a fluctuating or decreased estrogen level, a pharmaceutical composition comprising: azelastine, or a pharmaceutically acceptable salt of azelastine, and alprazolam. For example, a fluctuating or decreased estrogen level can be determined by measuring a first, second, and/or one or more subsequent estrogen levels of a patient, and comparing the first, second, and/or one or more subsequent estrogen levels of a patient to find whether the patient's estrogen level has increased and/or decreased from one measurement to another. If the level of estrogen of the patient decreased from one such measurement to another later performed measurement, then the patient has exhibited a decreased estrogen level. If the level of estrogen of the patient decreased then increased, or increased then decreased, from one such measurement to another later performed measurement, then the patient has exhibited a fluctuating estrogen level. Such comparisons can alternatively or in addition be made between a patient's estrogen level measurement and any reference or standard, such as an estrogen level typically expected for a patient of the same age. Further, for example, the comparison to determine whether a patient has exhibited a fluctuating or decreased estrogen level can involve determining a difference between the first estrogen level measurement and the second or any one or more subsequent estrogen level measurements, or between any two or more subsequent estrogen level measurements, or between the second and any one or more subsequent estrogen level measurements, or between any two or more of any of such estrogen level measurements, or between any of such estrogen level measurements and any estrogen level reference or standard.

Aspect 34 is the method of Aspect 33, wherein the pharmaceutical composition is administered once or twice a day, or once every 2 or 3 or 4 days, to the patient in an oral solid or liquid form.

Aspect 35 is the method of Aspect 33 or 34, wherein the pharmaceutical composition is administered for a period of at least 2 weeks.

Aspect 36 is the method of any of Aspects 33-35, wherein the azelastine or the pharmaceutically acceptable salt of azelastine is present in the pharmaceutical composition in an amount in the range of up to about 8 mg.

Aspect 37 is the method of any of Aspects 33-36, wherein the alprazolam is present in the pharmaceutical composition in an amount in the range of up to about 4 mg.

Aspect 38 is the method of any of Aspects 33-37, wherein the azelastine or the pharmaceutically acceptable salt of azelastine is present in the pharmaceutical composition in an amount that is 5 times the amount of alprazolam present in the pharmaceutical composition.

Aspect 39 is the method of any of Aspects 33-38, wherein the azelastine or the pharmaceutically acceptable salt of azelastine is present in the pharmaceutical composition in an amount of up to about 2 mg, and the alprazolam is present in the pharmaceutical composition in an amount of up to about 0.4 mg.

Aspect 40 is use of the pharmaceutical composition of any of Aspects 1-10 or Aspects 19-24 for the manufacture of a medicament for treating anxiety or depression associated with perimenopause, menopause, or fluctuating or decreased estrogen levels, or one or more symptoms thereof.

Aspect 41 is the pharmaceutical composition of any of Aspects 1-10 or Aspects 19-24 for use in the treatment of anxiety or depression associated with perimenopause, menopause, or fluctuating or decreased estrogen levels, or one or more symptoms thereof.

Aspect 42 is the pharmaceutical composition of any of Aspects 1-10 or Aspects 19-24 for use as a medicament.

Aspect 43 is the pharmaceutical composition of any of Aspects 1-10 or Aspects 19-24 for use as a medicament in the treatment of anxiety or depression associated with perimenopause, menopause, or fluctuating or decreased estrogen levels, or one or more symptoms thereof.

### DETAILED DESCRIPTION OF THE INVENTION

Through clinical practice, the inventors of the present invention found that a pharmaceutical composition with oral dosage forms comprising the active agents, a salt form of azelastine, and alprazolam, is suitable for treating perimenopausal or menopausal patients suffering from anxiety or depression.

The detailed description provided below is intended as a description of the present examples and is not intended to represent the only forms in which the present examples may be constructed or utilized. The description sets forth the functions of the examples and the sequence of steps for constructing and operating the examples. However, the same or equivalent functions and sequences may be accomplished by different examples.

### Definitions

As used in the present specification, the following words and phrases are generally intended to have the meanings as set forth below, except to the extent that the context in which they are used indicates otherwise.

As used herein, the term “alprazolam” refers to alprazolam free base, 8-Chloro-1-methyl-6-phenyl-4H-s-triazolo(4,3-a)(1,4)benzodiazepine.

As used herein, the term “azelastine” refers to azelastine free base, or 4-(p-Chlorobenzyl)-2-(hexahydro-1-methyl-1H-azepin-4-yl)-1-(2H)-phthalazinone. In certain embodiments, azelastine also includes any pharmaceutically acceptable salt, such as the hydrochloride or HCl salt. Preferably, in any embodiments of the invention as described herein, azelastine is in the form of its hydrochloride salt, as azelastine hydrochloride or azelastine HCl. More preferably, in any embodiment of the invention as described herein, reference to the amounts and dosage ranges of azelastine, for example in the solid oral dosage forms, are to the amounts and dosage ranges of azelastine hydrochloride, and reference to the amounts and dosage ranges of azelastine hydrochloride, for example in the solid oral dosage forms, are to the amounts and dosage ranges of azelastine or any pharmaceutically acceptable salt(s) thereof.

As used herein, “treating” or “treatment” means complete cure or incomplete cure, or it means that the symptoms of the underlying disease or associated conditions are at least affected, prevented, reduced, eliminated and/or delayed, and/or that one or more of the underlying cellular, physiological, or biochemical causes or mechanisms causing the symptoms are affected, prevented, reduced, delayed and/or eliminated. It is understood that reduced or delayed, as used in this context, means relative to the state of the untreated disease, including the molecular state of the untreated disease, not just the physiological state of the untreated disease.

The term “effective amount” refers to an amount that is sufficient to affect treatment, as defined below, when administered to an animal or mammal in need of such treatment. The therapeutically effective amount will vary depending upon the patient being treated, the weight and age of the patient, the severity of the disease condition, the manner of administration and the like, which can readily be determined by one of ordinary skill in the art. The pharmaceutical compositions may be administered in either single or multiple doses by oral administration. Administration may be by way of any one or more of capsule, tablet, gel, spray, drops, solution, suspensions, syrups, or the like.

The term “about” used herein in the context of quantitative measurements means the indicated amount ±10%. For example, with a ±10% range, “about 2 mg” can mean 1.8-2.2 mg.

The pharmaceutical compositions may be formulated for pharmaceutical use using methods known in the art, for example, Ansel's Pharmaceutical Dosage Forms and Drug Delivery Systems Tenth (by Loyd Allen, 2013) and Handbook of Pharmaceutical Manufacturing Formulations (Volumes 1-6 by Sarfaraz K. Niazi). Accordingly, incorporation of the active compounds and a controlled, or slow release matrix may be implemented.

Either fluid or solid unit dosage forms can be readily prepared for oral administration, for example, admixed with any one or more of conventional ingredients such as dicalcium phosphate, magnesium aluminum silicate, magnesium stearate, calcium sulfate, starch, talc, lactose, acacia, methyl cellulose and functionally similar materials as pharmaceutical excipients or carriers. A sustained release formulation may optionally be used. In older or incoherent subjects sustained release formulations may even be preferred. Capsules may be formulated by mixing the pharmaceutical composition with a pharmaceutical diluent which is inert and inserting this mixture into a hard gelatin capsule having the appropriate size. If soft capsules are desired, a slurry of the pharmaceutical composition with an acceptable vegetable, light petroleum or other inert oil can be encapsulated by forming into a gelatin capsule.

Suspensions, syrups and elixirs may be used for oral administration or fluid unit dosage forms. A fluid preparation including oil may be used for oil soluble forms. A vegetable oil such as corn oil, peanut oil or a flower oil, for example, together with flavoring agents, sweeteners and any preservatives produces an acceptable fluid preparation. A surfactant may be added to water to form a syrup for fluid unit dosages. Hydro-alcoholic pharmaceutical preparations may be used having an acceptable sweetener, such as sugar, saccharin or other non-nutritive sweetener, and/or a biological sweetener and/or a flavoring agent, such as in the form of an elixir.

The solid oral dosage formulation of this disclosure means a form of tablets, caplets, bi-layer tablets, film-coated tablets, pills, capsules, or the like. Tablets in accordance with this disclosure can be prepared by any mixing and tableting techniques that are well known in the pharmaceutical formulation industry. In some examples, the dosage formulation is fabricated by direct compressing the respectively prepared sustained-release portion and the immediate-release portion by punches and dies fitted to a rotary tableting press, ejection or compression molding or granulation followed by compression.

The pharmaceutical compositions provided in accordance with the present disclosure can be typically administered orally. This disclosure therefore provides pharmaceutical compositions that comprise a solid dispersion comprising azelastine and alprazolam as described herein and one or more pharmaceutically acceptable excipients or carriers including but not limited to, inert solid diluents and fillers, diluents, including sterile aqueous solution and various organic solvents, permeation enhancers, solubilizers, disintegrants, lubricants, binders, glidants, adjuvants, and combinations thereof. Such compositions are prepared in a manner well known in the pharmaceutical arts (see, e.g., Ansel's Pharmaceutical Dosage Forms and Drug Delivery Systems, Tenth (by Loyd Allen, 2013) and Handbook of Pharmaceutical Manufacturing Formulations (Volumes 1-6 by Sarfaraz K. Niazi)).

The pharmaceutical compositions may further comprise pharmaceutical excipients such as diluents, binders, fillers, glidants, disintegrants, lubricants, solubilizers, and combinations thereof. Some examples of suitable excipients are described herein. When the pharmaceutical compositions are formulated into tablets, tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

In embodiments, the pharmaceutical compositions can comprise a) about 1 mg-8 mg of azelastine HCl (or other salt thereof) and/or b) about 0.2 mg to 4 mg of alprazolam or a) about 2 mg-6 mg of azelastine HCl (or other salt thereof) and/or b) about 0.2 mg to 1 mg of alprazolam or a) about 2 mg-4 mg of azelastine HCl (or other salt thereof) and/or b) about 0.2 mg to 0.4 mg of alprazolam, or any amount of azelastine or alprazolam within these ranges. Additional embodiments include pharmaceutical compositions comprising a) about up to and including any of 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, or 8 mg azelastine, such as azelastine HCl, or any amount within any of these ranges and/or b) about up to and including any of 0.2 mg, 0.3 mg, 0.4 mg, 0.5 mg 0.6 mg, 0.7 mg, 0.8 mg, 0.9 mg, 1 mg, 1.25 mg, 1.5 mg, 1.75 mg, 2mg, 2.25 mg, 2.5 mg, 2.75 mg, 3 mg, 3.25 mg, 3.5 mg, 3.75 mg, or 4 mg alprazolam, or any salt thereof, or any amount within any of these ranges. For example, the compositions can comprise a) about 2 mg of azelastine HCl and/or b) about 0.4 mg of alprazolam HCl. Further, for example, compositions of the invention can comprise azelastine or a pharmaceutically acceptable salt of azelastine present in an amount in the range of about 1 mg to about 8 mg and alprazolam in an amount in the range of about 0.2 mg to about 4 mg. In embodiments, the amount of azelastine HCl (or other salt thereof) present in the composition can be equal to, more than, or less than the amount of alprazolam present in the composition. In embodiments, the amount of azelastine HCl (and/or other salt thereof) present in the composition can be 2 times as much, or 3 times as much, or 4, 5, 6, 7, 8, 9, 10, 15, 20, 25, 30, 35, 40, 45, or 50 times as much as the amount of alprazolam present in the composition, or vice versa. Any one or more of the compositions of the invention can be used with any one or more the methods of the invention disclosed herein, or other methods of using the compositions.

It will be understood, that the amount of the pharmaceutical composition containing azelastine HCl and alprazolam actually administered usually will be determined by a physician, in the light of the relevant circumstances, including the condition to be treated, the chosen route of administration, the actual compound administered and its relative activity, the age, weight and response of the individual patient, the severity of the patient's symptoms, and the like.

The pharmaceutical compositions, pharmaceutical dosage forms, and tablets containing azelastine, such as azelastine HCl, and alprazolam as described herein are administered to a perimenopausal or menopausal patient suffering from anxiety or depression, by administration (such as oral administration) once daily, twice daily, up to four times a day, once every other day, once a week, two times a week, three times a week, four times a week, or five times a week, or combinations thereof.

In embodiments, patients are administered the pharmaceutical composition(s) with a therapeutic effective daily dosage of azelastine or salt thereof (such as azelastine HCl) in the range of about 1 mg to about 8 mg and alprazolam or salt thereof in an amount in the range of about 0.2 mg to about 4 mg.

In embodiments, the pharmaceutical dosage forms and tablets of pharmaceutical compositions containing azelastine, such as azelastine HCl, and alprazolam as described herein are effective in affecting, preventing, reversing, reducing, alleviating, and/or treating one or more symptoms of anxiety and/or depression in perimenopausal or menopausal patients in about 1-8 weeks, such as within 1, 2, 3, 4, 5, 6, 7, or 8 weeks, or any range in between.

The following Example is illustrative and should not be interpreted to limit the scope of the claimed subject matter.

### EXAMPLE 1

A 43-year-old female patient had anxiety disorder for 7 months and was in perimenopause. She had feelings of panic attacks and fatigue at least once a day, showed excessive worries, extreme restlessness and irritability, and experienced insomnia 3-4 times a week. After taking alprazolam (2.0 mg/daily) for 1 month, she reported no satisfied improvement in any of her symptoms. Then she was treated with a composition of alprazolam of 0.4 mg and azelastine of 2 mg once daily. After 2 weeks, she exhibited dramatic clinical improvement and showed no symptoms of the anxiety disorder, including no panic attacks or fatigue. After an additional 4 weeks of treatment, she reported no excessive worries, restlessness, or irritability. Her insomnia was improved more than 70%. The inventors believe, in this case, the composition of two mechanisms of action for two different classes of diseases provided a new solution for treating her anxiety and depression caused by her perimenopause. Azelastine's anti-inflammatory action, which is achieved by suppressing release of cytokines, such as IL-1, IL-6, TNF-α and INF-α, in the CNS system may replace the diminished anti-inflammatory action of estrogen. Azelastine may also help alprazolam effectively bind to GABAA-benzodiazepine receptor complex which creates an effective presence of GABA to inhibit the action of several connected brain structures and further results in slowing of brain activity.

### REFERENCES

US Patent documents: U.S. Pat. No. 9,308,212, Apr. 12, 2016, Los; U.S. Pat. No. 8,865,733, Oct. 21, 2014, Felder; U.S. Pat. No. 8,168,625, May 1, 2012, Vandoni, et al; U.S. Pat. No. 9,919,050, Mar. 20, 2018, Dang, et al; U.S. Pat. No. 9,901,585, Feb. 27, 2018, Lulla, et al; U.S. Pat. No. 8,859,531, Oct. 14, 2014, Lee, et al; U.S. Pat. No. 8,758,816, Jun. 24, 2014, Fuge, et al; U.S. Pat. No. 8,318,709, Nov. 27, 2012, Lulla, et al; U.S. Pat. No. 8,304,405, Nov. 6, 2012, Lulla, et al; U.S. Pat. No. 8,168,620, May 1, 2012, Lulla, et al; U.S. Pat. No. 8,071,073, Dec. 6, 2011, Dang, et al; U.S. Pat. No. 7,022,687, Apr. 4, 2006, Szelenyl, et al; U.S. Pat. No. 6,017,909, Jan. 25, 2000, Hettche, et al; U.S. Pat. No. 5,994,357, Nov. 30, 1999, Theoharides; U.S. Pat. No. 5,086,050, Feb. 4, 1992, Hettche, et al; U.S. Pat. No. 5,068,233, Nov. 26, 1991, Achterrath-Tuckerman, et al.

Joris C. Verster and Edmund R. Volkerts, 2004. Clinical Pharmacology, Clinical Efficacy, and Behavioral Toxicity of Alprazolam: A Review of the Literature. CNS Drug Reviews, Vol. 10, No. 1, pp. 45-76.

Geoffrey K. Isbister, et al. 2004. Alprazolam is relatively more toxic than other benzodiazepines in overdose. British Journal of Clinical Pharmacology, Vol 58: 88-98.

J A Fawcett, H M Kravitz, 1982. Alprazolam: Pharmacokinetics, Clinical Efficacy, and Mechanism of Action. Pharmacotherapy, 2 (5): 243-54.

James P. Zacnya, Judith A. Paiceb, and Dennis W. Coalson, 2012. Separate and combined psychopharmacological effects of alprazolam and oxycodone in healthy volunteers, 2012 Aug. 1; 124(3): 274-282.

Danilo Nader and Linda Gowing, 2020. Is Long-Term Benzodiazepine Use a Risk Factor for Cognitive Decline? Results of a Systematic Review. J Addiction. January 23. doi: 10.1155/2020/1569456.

Edwards B J, Li J., 2013. Endocrinology of menopause. Periodontology 2000 61:177-194.

Giuliani N, et al., 2001. Serum interleukin-6, soluble interleukin-6 receptor and soluble gp130 exhibit different patterns of age- and menopause-related changes. Exp Gerontol 36:547-557.

Yasui T, et al., 2007. Changes in serum cytokine concentrations during the menopausal transition. Maturitas 56:396-403.

Pfeilschifter J., et al., 2002. Changes in proinflammatory cytokine activity after menopause. Endocr Rev 23:90-119.

Cushman M, et al., 1999. Effect of postmenopausal hormones on inflammation-sensitive proteins: The Postmenopausal Estrogen/Progestin Interventions (PEPI) Study. Circulation 100:717-722.

Simon F Crowe, Elizabeth K Stranks, 2018. The Residual Medium and Long-term Cognitive Effects of Benzodiazepine Use: An Updated Meta-analysis. Arch Clin Neuropsychol. 2018 Nov. 1; 33 (7): 901-911.

Ruihua Hou, 2017, Peripheral inflammatory cytokines and immune balance in Generalized Anxiety Disorder: case-controlled study. Brain Behav Immun. 2017 May; 62:212-218.

Michelle Guignet, 2020, Persistent behavior deficits, neuroinflammation, and oxidative stress in a rat model of acute organophosphate intoxication, Vol. 133, January 2020, 104431.

Chun-Hong Liu, 2019, Role of inflammation in depression relapse, Journal of Neuroinflammation (2019) 16:90 limit the scope of the claimed subject matter.

Jovana Vojvodic, et al, 2019, The Impact of Immunological Factors on Depression Treatment—Relation Between Antidepressants and Immunomodulation Agents, Macedonian Journal of Medical Sciences. 2019 Sep. 30; 7(18):3064-3069.

F. C. Bennett and A. V. Molofsky, 2019, The immune system and psychiatric disease: a basic science perspective, Clinical and Experimental Immunology, 197: 294-307.

Ruihua Hou and David S. Baldwin, 2012, A neuroimmunological perspective on anxiety disorders, Human Psychopharmacol Clin Exp. Vol 27: 6-14.

N Kappelmann, G Lewis, R Dantzer, P B Jones and G M Khandaker, 2018, Antidepressant activity of anti-cytokine treatment: a systematic review and meta-analysis of clinical trials of chronic inflammatory conditions, Molecular Psychiatry. Vol. 23, 335-343.

A. Atri, S. D. Rountree, O. L. Lopez, and R. S. Doody, 2018, Impact of Mast Cells in Depression Disorder: Inhibitory Effect of IL-37 (New Frontiers). Immunol Res, vol. 66 (3), 323-331 June 2018.

Sung Ho Maeng and Heeok Hong, 2019, Inflammation as the Potential Basis in Depression. Int Neurourol J 2019; Vol 23(Suppl 2): S63-71.

Ole Köhler, et al. van Hoven P T, Kaufman A, Carr W W. Inflammation in Depression and the Potential for Anti-Inflammatory Treatment. Current Neuropharmacology, 2016, 14, 732-742.

Anzela Niraula et al. 2019, Interleukin-6 Induced by Social Stress Promotes a Unique Transcriptional Signature in the Monocytes That Facilitate Anxiety. Biol Psychiatry 85 (8), 679-689 2019 Apr. 15.

M Catena-Dell'Osso, et al, 2011, Inflammatory and Neurodegenerative Pathways in Depression: A New Avenue for Antidepressant Development? Curr Med Chem. 18 (2), 245-55.

Duraisamy Kempuraj, et al. 2017, Mast Cell Activation in Brain Injury, Stress, and Post-traumatic Stress Disorder and Alzheimer's Disease, Front. Neurosci. 11:703. doi: 10.3389/fnins.2017.00703.

S Georgin-Lavialle et al. 2016, Mast Cells' Involvement in Inflammation Pathways Linked to Depression: Evidence in Mastocytosis. Mol Psychiatry. 21 (11), 1511-1516 November 2016.

Sang Won Jeon, Yong Ku Kim, 2016, Detrimental effect of preservative in eye drops: Neuroinflammation and cytokine abnormality in major depression: Cause or consequence in that illness? World Journal of Psychiatry, 2016 Sep. 22; 6(3): 283-293.

Romain Troubat et al, 2020, Neuroinflammation and Depression: A Review. Eur J Neurosci. 2020 Mar. 9 DOI: 10.1111/ejn.14720.

Ja Wook Kooa, et al, 2010, Nuclear factor-KB is a critical mediator of stressimpaired neurogenesis and depressive behavior. PNAS, Feb. 9, 2010, Vol. 107 (6) 2669-2674.

Patricia B Williams, Elizabeth Crandall and John D Sheppard, 2010, Azelastine hydrochloride, a dual-acting anti-inflammatory ophthalmic solution, for treatment of allergic conjunctivitis. Clinical Ophthalmology 2010:4 993-1001.

Casale T. The interaction of azelastine with human lung histamine H1, beta, and muscarinic receptor-binding sites. J Allergy Clin Immunol. 1989; 83:771-776.

Hazama H, Nakajima T, Hisada T, Hamada E, Omata M, Kurachi Y. Effects of azelastine on membrane currents in tracheal smooth muscle cells isolated from the guinea-pig. Eur J Pharmacol. 1994; 259: 143-150.

Szelenyi I, Achterrath-Tuckermann U, Schmidt J, Minker E, Paegelow I, Werner H. Azelastine: A multifaceted drug for asthma therapy. Agents Actions Suppl. 1991; 34:295-311.

Galatowicz G, Ajayi Y, Stern M E, Calder V L. Ocular antiallergic compounds selectively inhibit human mast cell cytokines in vitro and conjunctival cell infiltration in vivo. Clin Exp Allergy. 2007; 37:1648-1656.

Ciprandi G, Pronzato C, Passalacqua G, et al. Topical azelastine reduces eosinophil activation and intercellular adhesion molecule-1 expression on nasal epithelial cells: An antiallergic activity. J Allergy Clin Immunol. 1996; 98(6 Pt 1):1088-1096.

Simons F E, Simons K J. Clinical pharmacology of new histamine H1 receptor antagonist. Clin Pharmacokinet. 1999; 36:329-352.

A Aiko Hatakeyama, 2008, Azelastine hydrochloride on behavioral and psychological symptoms and activities of daily living in dementia patients. Geriatr Gerontol Int 2008; 8: 59-61.

Duraisamy Kempuraj, et al. 2003, Azelastine Inhibits Secretion of IL-6, TNF-alpha and IL-8 as Well as NF-kappaB Activation and Intracellular Calcium Ion Levels in Normal Human Mast Cells. Int Arch Allergy Immunol. 132 (3), 231-9 Nov 2003.

Kazunori Yoneda, et al. 1997, Suppression by Azelastine Hydrochloride of NF-KB Activation Involved in Generation of Cytokines and Nitric Oxide. Japanese Journal of Pharmacology, 73: 145-53.

Loyd Allen, Ansel's Pharmaceutical Dosage Forms and Drug Delivery Systems, Tenth (2013).

Sarfaraz K. Niazi, Handbook of Pharmaceutical Manufacturing Formulations Volumes 1-6.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Any of the methods disclosed herein can be used with any of the compositions disclosed herein or with any other compositions. Likewise, any of the disclosed compositions can be used with any of the methods disclosed herein or with any other methods. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1. A pharmaceutical composition, comprising:
up to about 8 mg azelastine or a pharmaceutically acceptable salt of azelastine;
alprazolam;
and one or more pharmaceutically acceptable excipients.

2. The pharmaceutical composition of claim 1, wherein the alprazolam is present in the pharmaceutical composition in an amount in the range of up to about 4 mg.

3. The pharmaceutical composition of claim 1, wherein the azelastine or the pharmaceutically acceptable salt of azelastine is present in the pharmaceutical composition in an amount 5 times that of the alprazolam present in the pharmaceutical composition.

4. The pharmaceutical composition of claim 3, wherein:
the azelastine or the pharmaceutically acceptable salt of azelastine is present in the pharmaceutical composition in an amount in the range of about 2 mg; and
the alprazolam is present in the pharmaceutical composition in an amount in the range of about 0.4 mg.

5. The pharmaceutical composition of claim 1, wherein the pharmaceutically acceptable salt of azelastine is azelastine hydrochloride.

6. The pharmaceutical composition of claim 1, wherein the pharmaceutical composition is formulated as an oral pharmaceutical dosage form.

7. A method comprising:
administering a pharmaceutical composition to a perimenopausal or menopausal patient;
wherein the pharmaceutical composition comprises azelastine, or a pharmaceutically acceptable salt of azelastine, and alprazolam.

8. The method of claim 7, wherein the pharmaceutical composition is administered once or twice a day, or once every 2 or 3 or 4 days, to the patient in an oral solid or liquid form.

9. The method of claim 8, wherein the pharmaceutical composition is administered for a period of at least 2 weeks.

10. The method of claim 7, wherein the azelastine or the pharmaceutically acceptable salt of azelastine is present in the pharmaceutical composition in an amount in the range of about 1 mg to about 8 mg.

11. The method of claim 10, wherein the azelastine or the pharmaceutically acceptable salt of azelastine is present in the pharmaceutical composition in an amount that is 5 times the amount of alprazolam present in the pharmaceutical composition.

12. The method of claim 10, wherein the alprazolam is present in the pharmaceutical composition in an amount in the range of about 0.2 mg to about 4 mg.

13. The method of claim 10, wherein the azelastine or the pharmaceutically acceptable salt of azelastine is present in the pharmaceutical composition in an amount in the range of about 1 mg to about 6 mg.

14. The method of claim 7, wherein:
the azelastine or the pharmaceutically acceptable salt of azelastine is present in the pharmaceutical composition in an amount in the range of up to about 8 mg; and
the alprazolam is present in the pharmaceutical composition in an amount in the range of up to about 4 mg.

15. A method comprising:
administering to a patient, who has exhibited a fluctuating or decreased estrogen level, a pharmaceutical composition comprising:
azelastine, or a pharmaceutically acceptable salt of azelastine; and
alprazolam.

16. The method of claim 15, wherein the pharmaceutical composition is administered once or twice a day, or once every 2 or 3 or 4 days to the patient in an oral solid or liquid form.

17. The method of claim 16, wherein the pharmaceutical composition is administered for a period of at least 2 weeks.

18. The method of claim 15, wherein the azelastine or the pharmaceutically acceptable salt of azelastine is present in the pharmaceutical composition in an amount in the range of up to about 8 mg.

19. The method of claim 18, wherein the alprazolam is present in the pharmaceutical composition in an amount in the range of up to about 4 mg.

20. The method of claim 15, wherein the azelastine or the pharmaceutically acceptable salt of azelastine is present in the pharmaceutical composition in an amount that is 5 times the amount of alprazolam present in the pharmaceutical composition.

21. The method of claim 20, wherein:
the azelastine or the pharmaceutically acceptable salt of azelastine is present in the pharmaceutical composition in an amount of up to about 2 mg; and
the alprazolam is present in the pharmaceutical composition in an amount of up to about 0.4 mg.
